(19) United States
(12) Patent Application Publication
(10) Pub. No.: US 2021/0024634 A1
(43) Pub. Date: Jan. 28, 2021

(54) METHODS FOR PREDICTING AND ENHANCING THERAPEUTIC BENEFIT FROM CHECKPOINT INHIBITORS IN CANCER

(71) Applicant: The Brigham and Women's Hospital, Inc., Boston, MA (US)

(72) Inventor: Hans Widlund, Brookline, MA (US)

(21) Appl. No.: 17/043,952

(22) PCT Filed: Mar. 29, 2019
(86) PCT No.: PCT/US2019/024992

§ 371 Date: Sep. 30, 2020

Related U.S. Application Data
(60) Provisional application No. 62/650,869, filed on Mar. 30, 2018.

(51) Int. Cl. C07K 16/28; A61P 35/00; A61K 45/06

(52) U.S. Cl. CPC C07K 16/2827; A61K 45/06; A61P 35/00; C07K 16/2818

(57) ABSTRACT

Methods for identifying those subjects who are most likely to benefit from treatment with a checkpoint inhibitor based on levels of CD200 and immune infiltrates, as well as methods for treating subjects with cancer using checkpoint inhibitors and MEK inhibitors.

### CLAIM OF PRIORITY

This application claims the benefit of U.S. Provisional Patent Application Ser. No. 62/650,869, filed on Mar. 30, 2019. The entire contents of the foregoing are hereby incorporated by reference.

### FEDERALLY SPONSORED RESEARCH OR DEVELOPMENT

This invention was made with Government support under Grant No. CA207982 awarded by the National Institutes of Health. The Government has certain rights in the invention.

### TECHNICAL FIELD

The present invention provides, in part, methods for identifying those subjects who are most likely to benefit from treatment with a checkpoint inhibitor based on levels of CD200 and immune infiltrates, as well as methods for treating subjects with cancer using checkpoint inhibitors alone, or in combination with BRAF and/or MEK targeted pharmacological inhibitors.

### BACKGROUND

Oncogene-targeted and immune-checkpoint therapies, respectively, have brought excitement and new hope to patients and physicians, but unfortunately have not achieved the ultimate goal of durable disease remission [1,2,3].

### SUMMARY

Therapeutic immune checkpoint blocking antibodies have revolutionized the treatment landscape for cancers, and now provide real hope to patients with advanced disease. While some patients will experience benefit, others will however not respond at all—a dilemma that is intimately linked to the genetic composition of the tumors. Identification of the molecular processes that enable, as well as preclude, positive responses to such therapies are urgently needed, and are likely to identify rational combinatorial approaches that will improve patient outcomes. Evidence has accumulated to indicate that a heightened mutational burden in the patients' tumors causes antigenicity through an increased frequency of cancer neoantigen peptides being displayed. In order to grow, such neoantigenic tumors must develop mechanisms that enable them to evade immune-mediated destruction, which makes these tumors specifically vulnerable to immune checkpoint therapies that targets aspects of this immune evasion. The present inventors have identified an immunosuppressive molecule, CD200, whose expression levels are linked to, and regulated by, inherent metabolic cues within the tumor cells that coincides with oncogenic NRAS and BRAF signaling. It is known that oncogene-targeted therapies aimed at BRAF(V600E) tend to cause an increased expression of melanoma antigens, which is driven by altered metabolic demands and governed by the melanoma-master regulator MITF and its transcriptional control of mitochondrial biogenesis through PGC1α. Expression of CD200 is positively regulated by the metabolic state governed by MITF and PGC1α, but also downstream of NRAS-BRAF-MEK signaling, and pharmacological inhibition of this signaling reduces its expression while coordinately increasing melanoma antigen expression. Hence, these data suggested a mechanism by which increased immune surveillance may intersect with the effects of targeted therapies. As shown herein, oncogenic signaling governs transcriptional regulation of CD200 transcription, and that manipulation of CD200 levels alters tumor growth in vivo, associated with changes in immunogenicity. Furthermore, heightened expression of CD200 is correlated with poor prognosis in patients who otherwise would be expected to benefit from immune checkpoint therapies.

Thus, provided herein are methods for determining whether a subject who has cancer is likely to benefit from treatment with a checkpoint inhibitor (e.g., for predicting whether a subject will respond to treatment with a checkpoint inhibitor). The methods include obtaining a sample comprising cancer cells from a subject; evaluating the presence and/or level of CD200 in the sample, and comparing the presence and/or level of CD200 with a reference level, wherein a level of CD200 that is less than or equal to the reference level of CD200 indicates a high likelihood of response and a level of CD200 in a subject that is greater than the reference level of CD200 indicates a low likelihood of response.

In some embodiments, the sample comprising cancer cells is obtained by punch biopsy, needle biopsy, or tissue biopsy obtained during resection.

In some embodiments, the methods include selecting a treatment comprising administration of a checkpoint inhibitor to a subject who has a level of CD200 that is less than or equal to the reference level of CD200.

In some embodiments, the methods include administering the treatment comprising administration of a checkpoint inhibitor to a subject who has a level of CD200 that is less than or equal to the reference level of CD200.

In some embodiments, the methods include selecting a treatment comprising administration of a checkpoint inhibitor and one or both of a MEK inhibitor and/or a BRAF inhibitor to a subject who has a level of CD200 that is greater than the reference level of CD200.

In some embodiments, the methods include administering the treatment comprising administration of a checkpoint inhibitor and one or both of a MEK inhibitor and/or a BRAF inhibitor to a subject who has a level of CD200 that is greater than the reference level of CD200.

In some embodiments, evaluating the presence and/or level of CD200 in the sample comprises determining a level of CD200 mRNA in the sample.

In some embodiments, the subject has melanoma, neuroblastoma, small cell lung carcinoma, mesothelioma, retinoblastoma, glioma, medulloblastoma, and ganglioneuroma. In some preferred embodiments, the subject has melanoma.

In some embodiments, the methods include determining whether the cancer is characterized by a high degree of immune infiltration, e.g., by (i) detecting the presence of NK, MDSC, CD4+, CD3+ and/or CD8+ immune cells; (ii) detecting the presence of PDL1 (programmed cell death ligand 1: CD274), PDL2 (programmed cell death ligand 2: PDCD1LG2), granzyme A (GMZA) and/or perforin transcripts (PRF1); and/or (iii) detecting the presence of PDL1, PDL2, granzyme A and/or perforin protein or activity. The detection of the presence of NK, MDSC, CD4+, CD3+ and/or CD8+ immune cells above a threshold; and/or levels or presence of PDL1, PDL2, granzyme A and/or perforin mRNA or protein above a reference level indicates the presence of immune infiltration and a high likelihood of response to checkpoint inhibitors, and levels or presence of PDL1, PDL2, granzyme A and/or perforin mRNA or protein above a reference level indicates the absence of immune infiltration and a low likelihood of response to checkpoint inhibitors.

In some embodiments, the methods include selecting a treatment comprising administration of a checkpoint inhibitor for a subject who has (i) NK, MDSC, CD4+, CD3+ and/or CD8+ immune cells above a threshold; and/or levels or presence of PDL1, PDL2, granzyme A and/or perforin mRNA or protein above a reference level, and (ii) a level of CD200 that is less than or equal to the reference level of CD200.

In some embodiments, the methods include administering the treatment comprising administration of a checkpoint inhibitor to a subject who has (i) NK, MDSC, CD4+, CD3+ and/or CD8+ immune cells above a threshold; and/or levels or presence of PDL1, PDL2, granzyme A and/or perforin mRNA or protein above a reference level, and (ii) a level of CD200 that is less than or equal to the reference level of CD200.

In some embodiments, the methods include selecting a treatment comprising administration of a checkpoint inhibitor and a MEK inhibitor to a subject who has (i) NK, MDSC, CD4+, CD3+ and/or CD8+ immune cells above a threshold; and/or levels or presence of PDL1, PDL2, granzyme A and/or perforin mRNA or protein above a reference level, and (ii) a level of CD200 that is greater than the reference level of CD200.

In some embodiments, the methods include administering the treatment comprising administration of a checkpoint inhibitor and a MEK inhibitor to a subject who has (i) NK, MDSC, CD4+, CD3+ and/or CD8+ immune cells above a threshold; and/or levels or presence of PDL1, PDL2, granzyme A and/or perforin mRNA or protein above a reference level, and (ii) a level of CD200 that is greater than the reference level of CD200.

In some embodiments, the methods include selecting a treatment that does not comprise administration of a checkpoint inhibitor to a subject who has levels of NK, MDSC, CD4+, CD3+ and/or CD8+ immune cells below a threshold; and/or levels of PDL1, PDL2, granzyme A and/or perforin mRNA or protein below a reference level.

In some embodiments, where immune cells are measured, only CD3+ cells are measured, or CD3+ cells and one or more of MDSC, NK, CD4+ and/or CD8+ cells.

In some embodiments, the selected treatment comprises one or both of a MEK inhibitor and/or a BRAF inhibitor.

Also provided herein are methods for treating a subject with cancer comprising administering to the subject a therapeutically effective amount of a checkpoint inhibitor and a MEK inhibitor. Also provided herein are a checkpoint inhibitor and a MEK inhibitor for use in treating a subject with cancer.

In some embodiments of the methods and compositions described herein, the MEK inhibitor is selected from the group consisting of trametinib, cobimetanib, Binimetinib (MEK162), Selumetinib, PD-325901, CI-1040, PD035901, U0126-EtOH, PD184352 (CI-1040), TAK-733, PD98059, PD318088, BI-847325, GDC-0623, APS-2-79 HCl, Myricetin, Honokiol, SL-327, Refametinib (RDEA119, Bay 86-9766), BIX 02189, BIX 02188, AZD8330, TAK-733, and Pimasertib. In some embodiments, the BRAF inhibitor is selected from the group consisting of BMS-908662, R05212054 (also known as RG7256 or PLX3603), GDC-0879, PLX-4720, GSK2118436, sorafenib tosylate, LGX818, vemurafenib, dabrafenib, encorafenib, or RAF265.

In some embodiments of the methods and compositions described herein, the checkpoint inhibitor is an antibody, preferably selected from the group consisting of anti-CD137; anti-PD-1 (programmed cell death 1); anti-PDL1; anti-PDL2; and anti-CTLA-4.

In some embodiments of the methods and compositions described herein, the subject has melanoma, neuroblastoma, small cell lung carcinoma, mesothelioma, retinoblastoma, glioma, medulloblastoma, and ganglioneuroma.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1B. Illustration of therapeutic benefit seen in patients from immune checkpoint treatments in a comparison to classical systemic therapies.

FIG. 2. Schematic illustration of how immune checkpoint therapies promote immune mediated elimination, resulting in tumor shrinkage or stable disease through resetting the tumor-immune equilibrium.

FIG. 3. In melanoma, intrinsic signaling and metabolic cues converge on C/EBPβ (or ETS) and YY1/PGC1α, respectively, to control CD200 levels. In turn, tumor expressed CD200 potentiates MDSCs bearing the CD200 receptor in the microenvironment to suppress CD8 T cells via PD1 ligands.

FIGS. 4A-4C. Analysis of TCGA data for stage IV melanoma (n=68) through segregation based on; 4A. PRF1:GZMA high/low, and 4B. PD-L1/L2 high/low. 4C. immunoedited tumors (defined as PRF1:GZMA high+PD-L1/L2 high) display significant prognosis stratification (HR=5.7) based on CD200 expression (p<0.015).

FIGS. 5A-5B. 5A. The immunoedited phenotype (high GZMA:PRF1 and PD-L1:L2) defines therapeutic benefit from anti-CTLA4 [15]. 5B. CD200 expression within immunoedited tumors trends towards precluded benefit from anti-CTLA4 therapy. Log Rank: n=40 in total cohort; and n=6 for each high/low CD200 group in the immunoedited cohort (high GZMA:PRF1 and PD-L1:L2).

FIGS. 6A-6D. Longitudinal measures of tumor volumes (W×W×L/2 mm3) following implantation of 1×10e5 cells of congenic YUMM1.7 melanoma cells with 6A) Cd200 over-expression and 6B) Cd200 shRNA in C57Bl/6J (wt) mice. The effects of modulation Cd200 levels by 6C) over-expression and 6D) shRNA suppression are void when implanted in B6/Cd200r1null mice. Significance (* with α=0.05) is based on one-way ANOVA across multiple comparisons.

FIG. 7. Schematic clinical decision matrix for treatment of patients based on baseline (prior-to-treatment) immune-infiltration and CD200 expression levels.

FIG. 8. Melanomas***, neuroblastomas***, small cell lung carcinomas***, and mesotheliomas* display disproportionally high expression of CD200 compared to other CCLE cell lines [64] based one-sided exact testing (observed vs. expected). ***p<0.001, *p<0.05.

FIGS. 9A-9B. 9A. Levels of CD200 across cell lineages identifies: the chromatin bound factors SUZ12 and MITF, and transcription factor binding motifs for MTF-1, CEBP, LEF1/TCF, HNF1A, ETS1. 9B. The CD200 flanking region contains conserved motifs for MITF, YY1, LEF/TCF, CEBP/ETS and a CpG island.

FIG. 10. Q-PCR in melanoma cell lines defined by increased NAD+/NADH redox status: A2058, A375, SKMEL5=low; UACC62, Malme3M, SKMEL28=high).

FIGS. 11A-C. 11A. CD200 decrease in melanomas after MEK-inhibitor treatment, but not in carcinomas (GSE10087). 11B. Consistent CD200 decrease following PD0325901 treatment (24 h; 100 nM). 11C. The pREP4-CD200 reporter recapitulates changes in response to MEK-inhibitor (24 h; 100 nM). Significance as * p<0.05: ** p<0.01, based on 2-sample, 2-tailed t-test.

FIG. 12. Dox-induced YY1-alleles (48 h: 100 ng/mL) activate CD200 and ESRRA/NDUFS1 expression in A2058 and YUMM1.1 cells. * p<0.05 for a 2-sample, 2-tailed t-test.

FIG. 13. Expression of Cd200 and NAD+/NADH ratios among B16, GL261, YUMM1.1 mouse lines.

FIGS. 14A-14B. 14A. Longitudinal growth of YUMM1.1 and YUMM1.7 in congenic C57Bl/6 animals (n=8/group; * indicates p<0.05 for two-sample, two-sided, t-test). 14B. qPCR of tumor-associated Cd200, GmzA and Prf1 expression in YUMM1 tumors.

FIGS. 15A-15E. 15A. Overexpression of Cd200 in YUMM1.7 leads to an increase in soluble Cd200 protein, and 15B. verified by ELISA from culture supernatant (rat monoclonal anti-mouse Cd200 and polyclonal anti-mouse Cd200 antibodies). 15C. Marginal effects on the cellular NAD+/NADH ratios can be observed following Cd200 over-expression. 15D. Cd200 promotes tumor growth in C57/BL6 animals. * indicates p<0.05 for a two-sided, two-sample t-test (n=8/cohort). 15E. Overexpression of Cd200 (>1000 fold***; 2-sample, 2-sided t test) in B16 cells do not measurably affect tumor growth kinetics (n=10 per cohort).

FIG. 16. Q-PCR analysis of tumors (Day 9) for expression of Cd200, PdL1, PdL2, and Prf1 as well as GzmA. There was an associated increase in PdL1 and PdL2 with Cd200, but not for Prf1 and GzmA. * p<0.05 based on 2-sample, 2-sided t-test, and ANOVA for groups.

FIG. 17. Analysis of Cd200 overexpression on the effect of immune checkpoint therapy. Change in tumor size was monitored by caliper measurements twice weekly.

### DETAILED DESCRIPTION

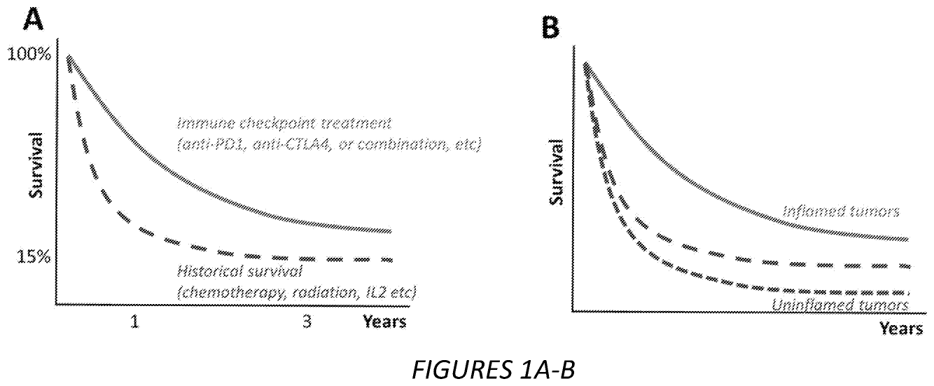

In contrast to most other human cancers, fatalities from malignant melanoma have been rising at a constant rate, and this malignancy disproportionally affects patients in the prime of their adult life [4]. Prior to recent improvements in melanoma therapy, the rapid progression to metastasis and the inherent resistance to conventional chemotherapies was the basis for a devastating prognosis of less than 50% survival one year after advanced-stage disease diagnosis, but recent progress in unleashing the immune system against tumors has yielded measurably improved survival (FIG. 1A). Specifically, 2011 marked approval for the first-in-class immune checkpoint inhibitor (anti-CTLA4, ipilimumab), which improves long-term patient survival [5,6]. Similarly, therapeutic antibodies targeting the PD1 receptor [7], such as pembrolizumab [8] and nivolumab [9], are thought to unleash PD1-bearing CD8 cell function in the tumor microenvironment via blocking PD1-ligands (PD-L1/L2) [10]. Anti-PD1 treatment was approved in 2015, and demonstrated improved benefit compared to anti-CTLA4 treatment [12]. To lengthen and broaden currently achieved treatment responses, there is an urgent need to understand barriers to their therapeutic efficacy—knowledge that critically will help to guide patient selection and inform the development of combinatorial approaches.

While the therapeutic advances in oncogene-targeted and immune checkpoint therapies have helped to extend lives for some patients, it is unclear who will respond to any given therapy. For example, not all patients whose melanomas carry BRAF(V600E) will benefit from BRAF-targeted therapeutics, and the marginal responses in non-melanoma BRAF(V600E)-cancers highlights that the presence of the mutant oncogene does not per se infer efficacy. Similarly, immune checkpoint inhibitors, represented by anti-PD1 and anti-CTLA4 biologicals, primarily benefits a subset of patients whose tumors harbor an elevated frequency of random missense mutations that yield neoantigens and are drivers of immune cell recognition (i.e. inflamed tumors) [11,12, 13, 14, 15]. Although one reason for infiltration by immune cells was only recently explained by neoantigen frequencies, inflamed tumors have long known to be associated with a generally better outcome (FIG. 1B).

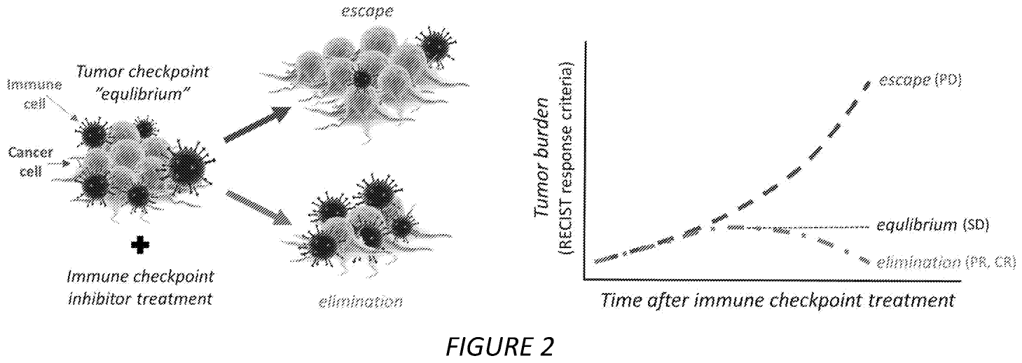

The concept of tumor immunoediting describes the evolutionary sculpting of tumors via immune-mediated cytolytic attrition of immunogenic tumors [16, 17, 18], which is central to understanding checkpoint therapy responses [19, 20, 21], and involves the steps of equilibrium, escape or elimination (FIG. 2). Checkpoint blockade responses are more likely in immunogenic cancers (neoantigen-rich tumors), whose presented mutant peptides have through growth sculpted the immunoedited tumor phenotype, which involves upregulation of immune suppressive PD1-ligands that inhibits the function of PD1-bearing CD8 cells [22, 23, 24, 25, 26]. Hence, tumor immunoediting involves upregulation of immune suppression (genes: CD274 & PDCD1LG2) in the tumor microenvironment to escape immune-mediated elimination (genes: PRF1 & GZMA).

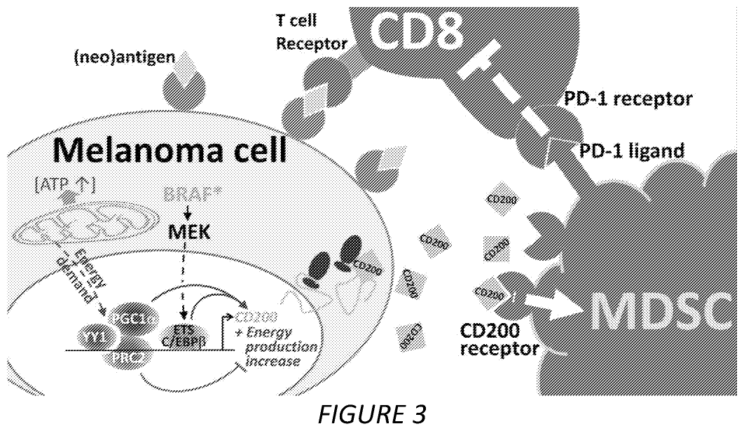

We recently defined a molecular link between MITF, the melanoma oncogene and melanocyte-differentiation master-regulator, and the control of cellular metabolism through the mitochondrial biogenesis co-activator PGC1α [60, 61]. Without wishing to be bound by theory, the present results support a model in which metabolic cues coordinately drive expression of the immune suppressive molecule CD200 (illustrated in FIG. 3). Increased CD200 may shape the tumor microenvironment by expanding CD200R1-bearing myeloid derived suppressor cells (MDSCs), which are known to coerce adaptive T cell immunity [27], and alter immune checkpoint inhibitor efficacy.

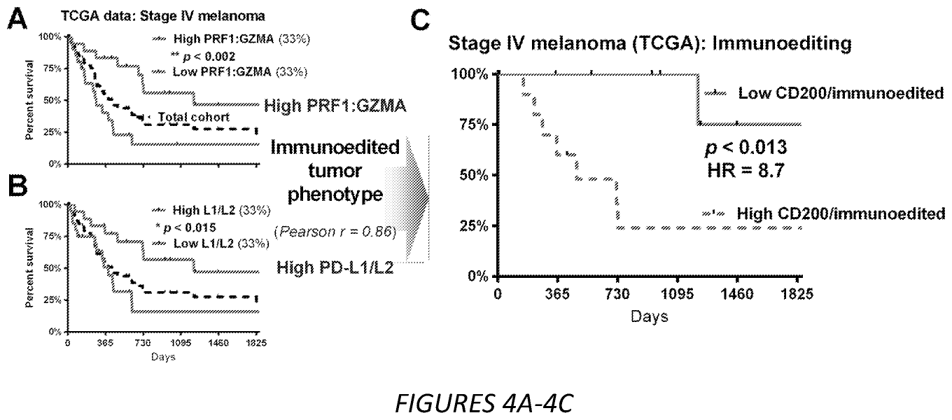

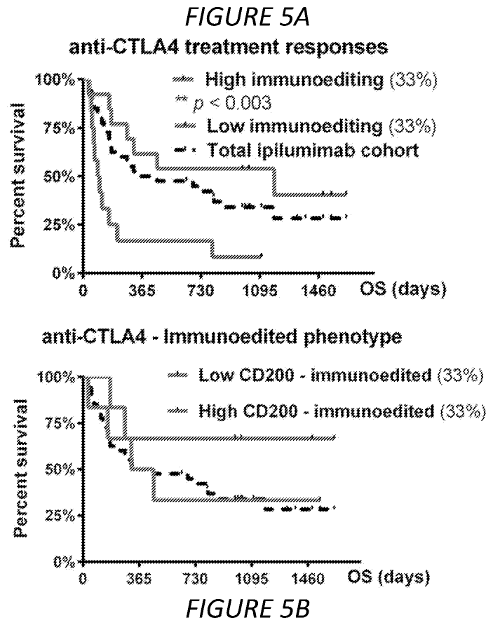

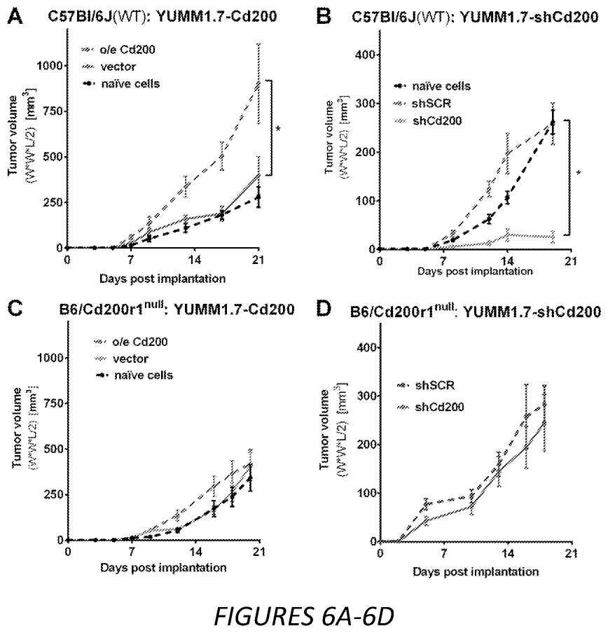

Specifically, the present inventors have found a striking trend between elevated CD200 and poor clinical outcomes for melanoma patients whose tumors display an inflamed “immunoedited” phenotype (high cytolytic index (GMZA:PRF1) and elevated PD1-ligands (CD274:PDCD1LG2) (illustrated in FIG. 4). Furthermore, within immunoedited tumors, high levels of CD200 limited patient treatment responses following CTLA4-antibody blockage (illustrated in FIG. 5). In addition, modulation of Cd200 expression in a mouse model of melanoma affected growth velocities, implicitly dependent on Cd200R1 (illustrated in FIG. 6).

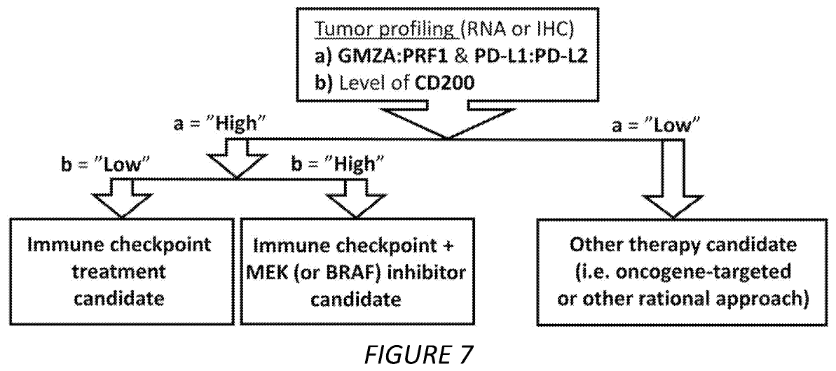

Hence, and as demonstrated herein, elevated CD200 represent an intrinsic means by which tumors modify the microenvironment—a paradigm that may prospectively be used to inform and enhance response to immune checkpoint inhibitors. The present data suggested CD200 as a central immune evasive molecule in a defined set of tumors, which can be used to prospectively inform immune checkpoint treatment benefit in the clinic, as shown in FIG. 7.

Thus, described herein are methods for identifying subjects who would be most likely to benefit from treatment with a checkpoint inhibitor, as well as methods for enhancing the likelihood of receiving benefit from such treatment.

Subjects

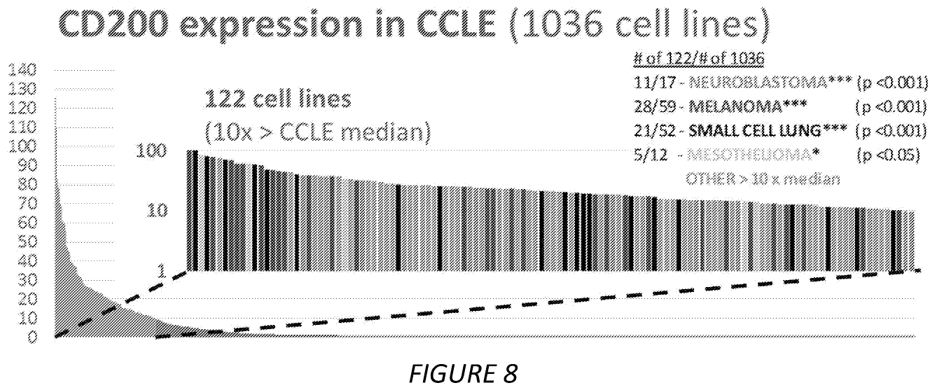

The present methods can be used in the selection and treatment of subjects with cancer, particularly those subjects with cancers of neuroectodermal origin, e.g., melanomas, neuroblastomas, small cell lung carcinomas, and mesotheliomas (FIG. 8), as well as retinoblastomas, gliomas, medulloblastomas, and ganglioneuromas, and other cancers with high levels of CD200. These are malignant neoplasms arising in the neuroectoderm, the portion of the ectoderm of the early embryo that gives rise to the central and peripheral nervous systems. Methods for identifying or diagnosing subjects with a cancer are known in the art, and can include biopsy, imaging, and biomarker analysis.

Methods for Identifying and Selecting Subjects for Treatment

Described herein are methods for identifying those subjects who are most likely to benefit from treatment with a checkpoint inhibitor. The methods include determining levels of CD200 expression (e.g., NCBI RefSeq ID NM_005944.6 and/or NM_001004196.3, the two longest isoforms that are the highest expressed and contain all common exons) in cancer cells from the subject, e.g., cancer cells obtained by biopsy, e.g., punch biopsy, needle biopsy, or tissue biopsy obtained during resection.

The methods include obtaining a sample from a subject, and evaluating the presence and/or level of CD200 in the sample, and comparing the presence and/or level of CD200 with one or more references. Although gene expression of CD200 (e.g., mRNA levels) is exemplified herein, protein levels can also be used, e.g., levels of soluble CD200 protein (e.g., NCBI RefSeq ID NP 001004196.2 or NP 005935.4).

The methods can also include determining whether the cancer is characterized by a high degree of immune infiltration. Methods for detecting immune infiltration include performing histopathology to detect the presence of total CD3+, or CD8+ and CD4+, NK1, or specific immune cell subsets, and/or detecting the presence of genes associated with cytolytic cells, e.g., natural killer (NK) or CD8+ cells, e.g., such as based on the detection of gene expression of CD3 (CD3D: e.g., NCBI RefSeq ID. NM_000732.3, CD3E: e.g., NCBI RefSeq ID. NM_000733.3, CD3G: e.g., NCBI RefSeq ID. NM_000073.2) and/or cytolytic killing potential through granzyme A (e.g., NCBI RefSeq ID. NM_006144.3) and/or perforin 1 (e.g., NCBI RefSeq ID. NM_005041.5 and/or NM_001083116.2) (e.g., detection of transcripts, e.g., using RT-PCR); and/or detection of cytolytic activity associated with the tumor based on the detection of protein levels of granzyme A and/or perforin (e.g., detection of protein, e.g., using immunohistochemistry (IHC). The detection of CD3+, CD8+, or other immune cells; and/or levels or presence of granzyme A and/or perforin mRNA or protein above a reference level, indicates the presence of immune infiltration (e.g., Johnson et al., Proceedings of the National Academy of Sciences of the United States of America. 2003; 100:2657-2662; Schumacher et al., Cancer research. 2001; 61:3932-3936). The presence of immune infiltration is associated with a higher likelihood of response to checkpoint inhibitors (Ji et al., Cancer immunology, immunotherapy: CII. 2012; 61:1019-1031).

Suitable reference values can be determined using methods known in the art, e.g., using standard clinical trial methodology and statistical analysis. The reference values can have any relevant form. In some cases, the reference comprises a predetermined value for a meaningful level of CD200, e.g., a reference corresponding to a level of CD200 in a representative subject or cohort of subjects that is sensitive to checkpoint inhibitors, and/or a level of CD200 in a representative subject or cohort of subjects that is sensitive to checkpoint inhibitors. Suitable reference levels of granzyme A, perforin, and immune cells can also be determined.

The predetermined or reference level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with response to checkpoint inhibitors in one defined group is a fold higher, or lower, (e.g., approximately 2-fold, 4-fold, 8-fold, 16-fold or more) than the response to checkpoint inhibitors in another defined group. It can be a range, for example, where a population of subjects (e.g., control subjects) is divided equally (or unequally) into groups, such as a low-likelihood of response group, a medium-likelihood of response group and a high-likelihood of response group, or into quartiles, the lowest quartile being subjects with the lowest likelihood of response and the highest quartile being subjects with the highest likelihood of response, or into n-quantiles (i.e., n regularly spaced intervals) the lowest of the n-quantiles being subjects with the lowest likelihood of response and the highest of the n-quantiles being subjects with the highest likelihood of response.

Thus, in some cases the level of CD200 in a subject being less than or equal to a reference level of CD200 is indicative of a high likelihood of response. In other cases the level of CD200 in a subject being greater than or equal to the reference level of CD200 is indicative of a low likelihood of response. In some embodiments, the amount by which the level in the subject is less (or more) than the reference level is sufficient to distinguish a subject from a control subject, and optionally is a statistically significantly less (or more) than the level in a control subject. In cases where the level of CD200 in a subject being equal to the reference level of CD200 the “being equal” refers to being approximately equal (e.g., not statistically different).

The predetermined value can depend upon the particular population of subjects (e.g., human subjects) selected. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample are first isolated according to standard methods, for example using lytic enzymes, chemical solutions, or isolated by nucleic acid-binding resins following the manufacturer's instructions.

The presence and/or level of a nucleic acid can be evaluated using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook, et al, Molecular Cloning: A Laboratory Manual (3. Sup.rd Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of CD200. Measurement of the level of a biomarker can be direct or indirect. For example, the abundance levels of CD200 can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such actin B (ACTB (e.g., NM_001101.4)), glyceraldehyde dehydrogenase (GAPDH (e.g., NM_002046.6)) and RPLPO (36B4, e.g., NM_001002.3), are most commonly used.

Gene arrays are prepared by selecting probes that comprise a polynucleotide sequence, and then immobilizing such probes to a solid support or surface. For example, the probes can comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

The presence and/or level of a protein can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a mictrotiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of proteins (See U.S. Pat. Nos. 5,118,937; 5,045,694; 5,719,060; 6,225,047).

In some embodiments, the methods include selecting subjects for treatment using a checkpoint inhibitor using a decision tree as shown in FIG. 7. Specifically, baseline levels of immune-infiltration and/or CD200 levels are measured (based on measurement method, each compared to a certain predetermined standard) in patient tumor biopsies, and treatment selected based on a) high/low immune infiltration, b) high/low CD200 levels; wherein high immune-infiltration and high CD200 would be an indicative of suitability for combinatorial treatment using immune checkpoint blocking antibody(-ies) and BRAF/MEK inhibitor.

Checkpoint Inhibitors

Immune checkpoint blockade has shown remarkable results in certain cancers and patient groups; currently approved immune checkpoint blockers are monoclonocal antibodies (mAbs) that target the programmed cell death protein 1 (PD-1)/PD-L1/2 or cytotoxic T-lymphocyte-associated protein 4 (CTLA-4) pathways, and agents targeting other pathways are in clinical development (including OX40, Tim-3, and LAG-3) (See, e.g., Leach et al., Science 271, 1734-1736 (1996); Pardoll, Nat. Rev. Cancer 12, 252-264 (2012); Topalian et al., Cancer Cell 27, 450-461 (2015); Mahoney et al., Nat Rev Drug Discov 14, 561-584 (2015)). The present methods can include the administration of checkpoint inhibitors such as antibodies including anti-CD137 (BMS-663513); anti-PD-1 (programmed cell death 1) antibodies (including those described in U.S. Pat. Nos. 8,008,449; 9,073,994; and US20110271358, pembrolizumab, nivolumab, Pidilizumab (CT-011), BGB-A317, MEDI0680, BMS-936558 (ONO-4538)); anti-PDL1 (programmed cell death ligand 1) or anti-PDL2 (e.g., BMS-936559, MPDL3280A, atezolizumab, avelumab and durvalumab); or anti-CTLA-4 (e.g., ipilumimab or tremelimumab). See, e.g., Kruger et al., “Immune based therapies in cancer,” Histol Histopathol. 2007 June; 22(6):687-96; Eggermont et al., “Anti-CTLA-4 antibody adjuvant therapy in melanoma,” Semin Oncol. 2010 October; 37(5):455-9; Klinke D J 2nd, “A multiscale systems perspective on cancer, immunotherapy, and Interleukin-12,” Mol Cancer. 2010 Sep. 15; 9:242; Alexandrescu et al., “Immunotherapy for melanoma: current status and perspectives,” J Immunother. 2010 July-August; 33(6):570-90; Moschella et al., “Combination strategies for enhancing the efficacy of immunotherapy in cancer patients,” Ann N Y Acad Sci. 2010 April; 1194:169-78; Ganesan and Bakhshi, “Systemic therapy for melanoma,” Natl Med J India. 2010 January-February; 23(1):21-7; Golovina and Vonderheide, “Regulatory T cells: overcoming suppression of T-cell immunity,” Cancer J. 2010 July-August; 16(4):342-7.

In some embodiments the methods include administering an immunotherapy selected from the group consisting of BiovaxID (an autologous vaccine containing tumor-specific idiotype proteins from individual patient's lymphoma cells conjugated to keyhole limpet hemocyanin (KLH)); Provenge sipuleucel-T (an FDA-approved example of the use of autologous dendritic cells); IMA901 (a vaccine containing 10 tumor-associated peptides (TUMAPs)), alone or in combination with Sutent (a small molecule VEGF receptor tyrosine kinase inhibitor); GV1001 (a peptide vaccine with the sequence of human telomerase reverse transcriptase (hTERT), from Kael-Gemvax); Stimuvax (a liposomal vaccine containing a synthetic 25-amino acid peptide sequence from mucin 1 (MUC1; CD227)); ISF35 or Lucatumumab (HCD122) (mAbs against CD40); GVAX (an allogeneic cancer vaccine engineered to secrete granulocyte macrophage-colony stimulating factor (GM-CSF)). See, e.g., Flanagan, “Immune Springboard,” Biocentury, Jun. 18, 2012 A5-A10 (2012), available at biocentury.com. In some embodiments, the immunotherapy comprises administration of an agent that effects CTLA4 blockade (e.g., Ipilumumab BMS), PD1-blockade (e.g., BMS-936558, BMS; CT-011, Curetech; MK-3475, Merck), CD137 activation (e.g., BMS-663513, BMS), PD-L1 blockade (e.g., BMS-936559, BMS), CD40 activation (e.g., CP-870893, Pfizer), and/or autologous dendritic cells (e.g., Provenge).

Methods for Treating Subjects

The methods described herein can thus include the use of pharmaceutical compositions comprising a checkpoint inhibitor, e.g., anti-PD1, and as the active ingredient. In addition, as shown herein, inhibiting MEK or upstream oncogenic BRAF results in a decrease in CD200 levels. Thus, the methods can include administration of a checkpoint inhibitor, e.g., anti-PD1, with a MEK inhibitor, e.g., trametinib, cobimetanib, binimetinib (MEK162), selumetinib, PD-325901, CI-1040, PD035901, U0126-EtOH, PD184352 (CI-1040), TAK-733, PD98059, PD318088, BI-847325, GDC-0623, APS-2-79 HCl, Myricetin, Honokiol, SL-327, refametinib (RDEA119, Bay 86-9766), BIX 02189, BIX 02188, AZD8330, TAK-733, and/or pimasertib (AS-703026), or a BRAF inhibitor, e.g. BMS-908662, R05212054 (also known as RG7256 or PLX3603), GDC-0879, PLX-4720, GSK2118436, sorafenib tosylate, LGX818, vemurafenib, dabrafenib, encorafenib, or RAF265 (see, e.g. Morris and Kopetz, F1000Prime Rep. 2013; 5:11), or a combination of BRAF plus MEK inhibitors. In some embodiments, the checkpoint inhibitor and MEK (or BRAF or BRAF/MEK combination) inhibitor(s) can be administered together (e.g., in the same composition, or at substantially the same time, e.g., within one minute of each other) or consecutively (e.g., one after the other, e.g., within two, 5, 10, 15, 20, 30, 60, or 120 minutes or more of each other). In some embodiments they are administered on the same day, within a few (2-3) hours of each other, or in parallel over the course of weeks and/or months. For example, the BRAF or MEK inhibitors can be taken orally daily, while the immune checkpoint inhibitors are typically infused on a three week schedule

Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., a MEK inhibitor. Pharmaceutical compositions are typically formulated to be compatible with its intended route of administration. Examples of routes of administration include parenteral, e.g., intravenous, intradermal, subcutaneous, oral (e.g., inhalation), transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELTM (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof.

Therapeutic compounds that are or include nucleic acids can be administered by any method suitable for administration of nucleic acid agents, such as a DNA vaccine. These methods include gene guns, bio injectors, and skin patches as well as needle-free methods such as the micro-particle DNA vaccine technology disclosed in U.S. Pat. No. 6,194,389, and the mammalian transdermal needle-free vaccination with powder-form vaccine as disclosed in U.S. Pat. No. 6,168,587. Additionally, intranasal delivery is possible, as described in, inter alia, Hamajima et al., Clin. Immunol. Immunopathol., 88(2), 205-10 (1998). Liposomes (e.g., as described in U.S. Pat. No. 6,472,375) and microencapsulation can also be used. Biodegradable targetable microparticle delivery systems can also be used (e.g., as described in U.S. Pat. No. 6,471,996).

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

### EXAMPLES

The invention is further described in the following examples, which do not limit the scope of the invention described in the claims.

CD200 is thought to suppress immune-surveillance through myeloid derived suppressor cells (MDSCs), which interfere with innate and adaptive immune-mediated tumor cell killing [28]. Furthermore, CD200 is a membrane glycoprotein identified in neurons that does not signal within the expressing cell [29]. Without wishing to be bound by theory, this integrated analysis is believed to have identified a direct link between the metabolic state of tumors and their immune evasive potential through CD200. This may help to explain how certain tumors co-exist within a hostile immune microenvironment and to understand immune checkpoint responses.

### Example 1—Intrinsic Mechanisms Regulating CD200 and its Function in Immune Evasion

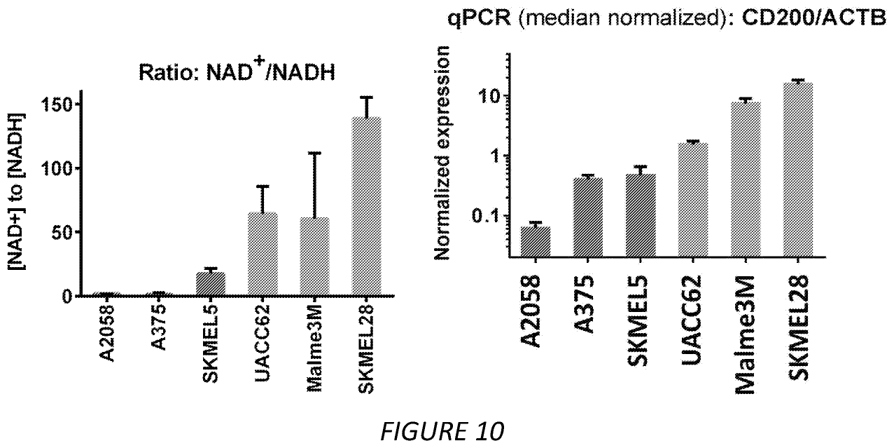

CD200 is an immune suppressive molecule that is associated with poor prognosis in certain solid cancers and hematologic disorders [30, 31], and among the most differentially expressed genes across melanoma cells stratified by their inherent metabolic state (FIG. 10). Antibody-targeting of CD200 has been shown to have anti-cancer effects in melanoma [32], CLL [33], and other cancer models [28]. It would therefore be advantageous to characterize (4A) how melanomas regulate CD200 expression, to assess (4B) effects on tumor growth, and to identify (4C) the target CD200-receptor bearing cells.

### Example 2A—Regulation of CD200 Expression

Because some melanomas [32] and other tumors [33] have elevated expression of CD200, we interrogated the Cancer Cell Line Encyclopedia (CCLE [34]) to examine expression patterns across multiple cancer cell lines (FIG. 8). We found that in addition to melanomas, also neuroblastoma, small cell lung carcinomas, and to lesser extent mesotheliomas, display a disproportionately elevated CD200 expression (10×>median). Because all are of neuroectodermal origin, it suggests that developmental cues may regulate CD200 expression.

(2A-1) Metabolic Cues Converging on Regulating CD200 Expression—

We assessed CD200 expression levels across a cohort of melanoma cell lines annotated for their NAD+/NADH ratios (FIG. 10), which results are in agreement with publicly available data [FIG. 8; 34].

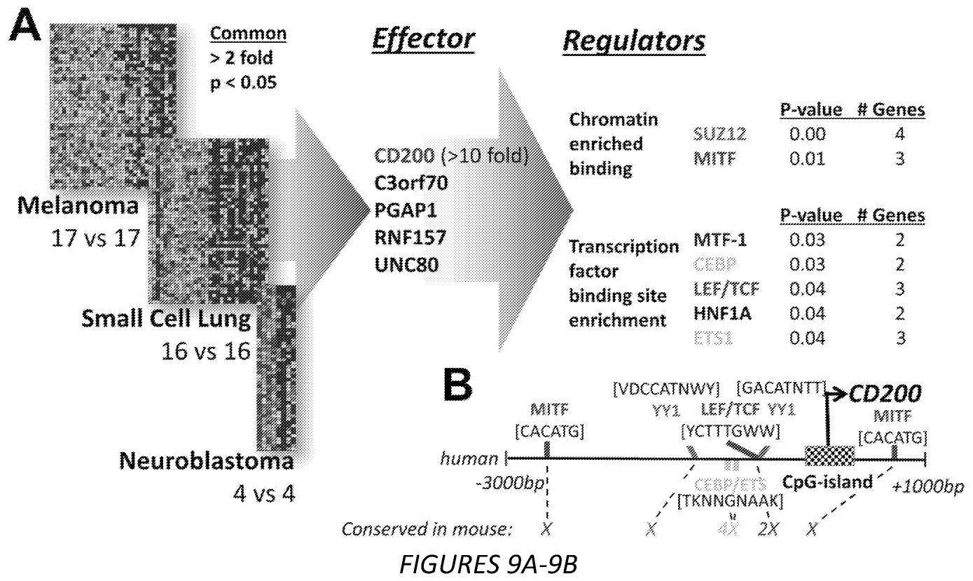

To extend this analysis and to search for common regulators, we compared CD200 expression across melanoma, small cell lung carcinoma, and neuroblastoma (FIG. 9A). Using a stringent cut-off (>2 fold expression; p<0.05 by 2-sided, 2-sample t test), we defined genes that are co-regulated along with CD200, and used these to identify prospective chromatin bound upstream regulators and transcription factor motifs (p<0.05 and >1 gene bound) using the Enrichr analysis tool [35]. While this analysis underscores MITF as a likely direct regulator of CD200 in melanoma [36, 37], it also identified the chromatin regulator SUZ12 (polycomb group repressor PRC2) and binding motifs for the pioneering HMG-factors LEF1/TCF, and the MAPK activated C/EBP and ETS1 proteins (FIG. 9B). Without wishing to be bound by theory, the relationship among these factors and functional relevance on regulating CD200 can be summarized accordingly;
YY1 recruits the repressive polycomb complex PRC2 that contain SUZ12 to alter chromatin structure [38];
Phosphorylation of YY1 facilitates interaction with PGC1a to activate genes involved in mitochondrial biogenesis [39, 40];
Polycomb group complexes [41] and PGC1a regulate responses to oxidative stress [60];
The proximal CD200 promoter drives expression and is controlled by C/EBPβ [42];
C/EBP and ETS factors phosphorylated by MAPK, which activates their function [43, 44, 45].

Specifically in melanocytes and melanoma,
YY1 and MITF [46, 47], as well as LEF1 and MITF [48], coordinately regulate lineage-specific transcription;
The dual E-box motifs (CATGTG) in CD200 resembles the pigment genes: TYR, TYRP, and DCT [49];
CD200 expression is elevated and maintained by the MAPK pathway [32].

Consequently, the CD200 flanking region contains sequence motifs that are functionally implied (FIG. 9B). Moreover, surrounding the transcription start is a CpG-island that may confer epigenetic control of transcription directed by the PRC2 complex. With emphasis on the convergence of C/EBP:ETS with MAPK-activity and YY1 interactions with PGC1α or PRC2, we will assess how signaling and bioenergetic demand coordinately control CD200 levels.

The region next to the CD200 transcription start drives basal activity [42]. We have therefore amplified an 850 bp fragment upstream of the CD200 start codon, and cloned it into an EBNA-carrying vector (pREP4-LUC/hygromycin [50]) that remains episomal and replicates within human cells and allows nucleosome deposition to form chromatin.

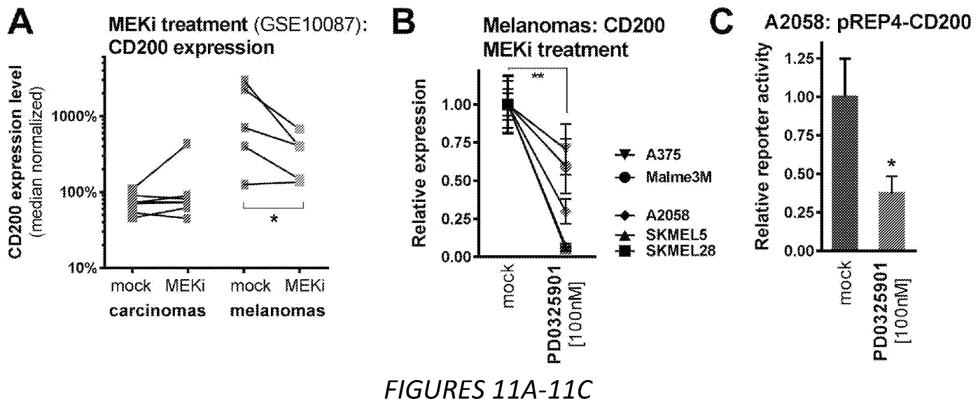

Because CD200 has been identified as regulated by MAPK-signaling in melanoma cells [32], a pathway which is frequently deregulated by BRAF(V600E) function [4, 62], we wanted to assure that this mode of regulation holds true. To this end, we analyzed publicly available data (GSE10087) on MEK-inhibitor treatment across a cohort of carcinoma and melanoma cell lines (FIG. 11A) [51], measured CD200 levels by qPCR following MEK inhibition in a cohort of our melanoma cell lines (FIG. 11B), and initial reporter assays (FIG. 11C). We found that CD200 levels consistently are reduced in melanoma cell lines following PD0325901 treatment, and the pREP4-CD200 reporter parallels these responses.

Using qPCR, we measured the effect of MEK-inhibitor treatment on CD200 RNA levels across our panel of melanoma cell lines (see FIG. 11B), using 24 h treatment with PD0325901 [100 nM]. Specifically, we found a consistent downregulation of CD200 levels following MEK-inhibitor treatment.

(2A-2) YY1 Regulation of CD200 and Coordination with Metabolic Demands—

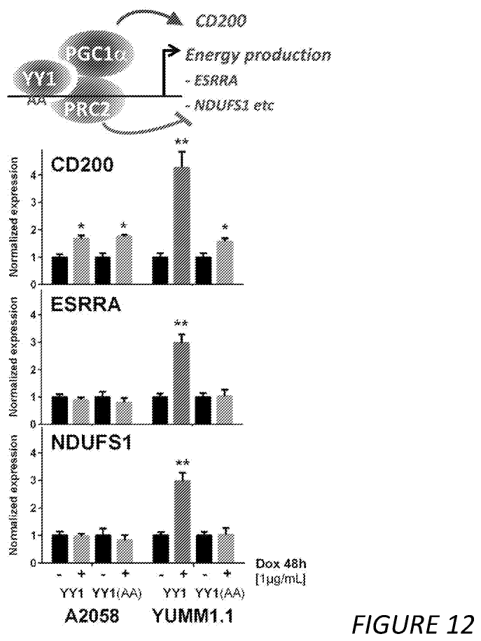

There are multiple YY1 binding sites in the CD200 regulatory region (FIG. 9), and the polycomb group protein YY1, can either act as a repressor of transcription through recruitment of the PRC2 complex (containing SUZ12) [38], or as an activator with PGC1α to promote expression of genes involved in mitochondrial biogenesis [39, 40]. We examine whether YY1 represses or activates CD200 expression in melanoma cells. Using doxycycline-inducible lentiviral constructs (pIND20-DEST.neo), we have established melanoma cells with YY1 or YY1(AA) (Blättler et al., Cell Metab. 2012 Apr. 4; 15(4):505-17). The YY1(AA)-allele has reduced ability to interact with PGC1α, and thus is less able to transactivate genes involved in mitochondrial biogenesis [40]. Importantly, upon administration of doxycycline for 48 h, the levels of CD200 are increased; however in a manner that is separable between YY1(AA) and downstream effects on mitochondrial biogenesis genes—ESRRA, NDUFS1 (FIG. 12). Based on these data, YY1 seems to have two functions; YY1/YY1(AA) promotes basal CD200 transcription, and along with mitochondrial biogenesis genes, YY1-PGC1α activates CD200.

### Example 2B—Examine the Relationship Between CD200 and Tumor Phenotypes

To determine whether CD200 levels influence patient outcome, we analyzed The Cancer Genome Atlas (TCGA) melanoma data [11] (FIG. 4). Killing of cancer cells by cytolytic immune cells (CD8+T and natural killer—NK) cells involves granzyme A (GZMA) and perforin-1 (PRF1) [52], and this defines inflamed tumors that predict better long-term prognosis for cancer patients [53]. Furthermore, upregulation of immune-suppressive ligands, such as PD-L1 and PD-L2 in the tumor microenvironment, is central to PD1-mediated immune suppression and explains how cancers coexist with surveillance [20]. Because of the high correlation (Pearson r=0.86) between GZMA:PRF1 (a composite metric) and PD-L1/L2 expression across advanced stage melanomas, each which stratifies outcome; p<0.002 and p<0.015, respectively, we have chosen these in aggregate to define the “immunoedited phenotype” of tumors. Within this cohort of “inflamed” tumors that co-exist with immune cell attrition, there was a striking association between poor outcome and high CD200 expression (FIG. 4C). Although this cohort contains some patients that have received BRAF- and/or immune checkpoint therapies, they are too few to affect the analysis.

Considerations for Mouse Tumor Immunogenicity Studies—

To study tumor and immune cell interactions in vivo, there is an implicit need for an intact immune system, which generally precludes studies of human cancer cells in animal models. Animal tumors allow studies in their syngeneic hosts; however, spontaneous tumor models are often of low immunogenic grade, such as the B16 melanoma cell line from a C57Bl/6 host [54]. In contrast, chemically or virally induced cancers are often immunogenic, such as methylcholanthrene-induced sarcomas, and was the study of these cancers that launched the concept of immunoediting [20]. As a complement, transgene-mediated oncogenesis in the mouse has proven useful, where the melanoma model: tg.Braf(V600E)/Pten^{null}/Tyr.CRE^{tm} [29] has provided insight to immunosuppression that involves Tregs, MDSCs [55], and modifiable by immunotherapy [63, 64].

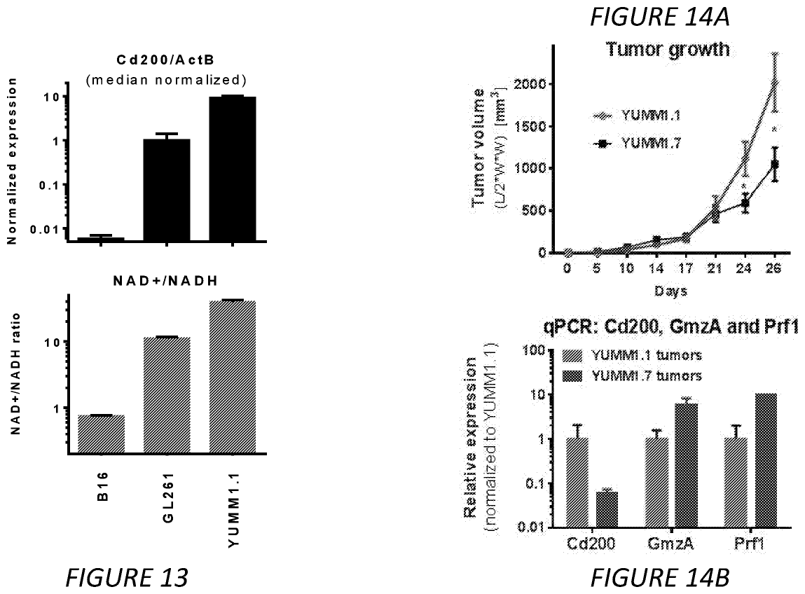

We determined Cd200 mRNA levels and assessed NAD+/NADH ratios in three mouse cancer cell lines, including the mouse glioma model GL261 (FIG. 13). There was a clear trend between Cd200 expression levels and the intrinsic metabolic state in these different mouse cancer cells. In particular, the YUMM1 cell line(s), which are derived from primary tg.Braf(V600E)/Cdkn2a^{null}/Pten^{null}/tyr.CRE^{tm} melanoma tumors exhibited heightened Cd200 expression and therefore are suitable for our experimental uses in vivo.

(2B-1) Modulation of Cd200 Levels on Tumor Growth and the Associated Immune Phenotype

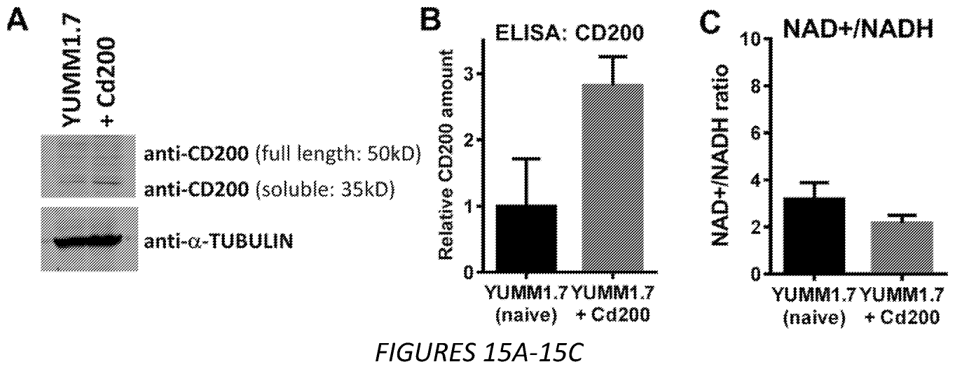

To study the relationship between Cd200 expression and tumor growth, we used independently isolated melanoma cell lines from the Bosenberg laboratory—YUMM (FIG. 14A). Interestingly, levels of Cd200 and infiltrating cytolytic immune cells—measured as granzyme A (GmzA) and perforin (Prf1) levels, correlated with the observed growth rate differences between these two cell lines (FIG. 14B).

These data provide some evidence that levels of Cd200 may affect in vivo growth of these tumors that parallels experimental assessment of the clinical correlates in patients regarding tumor microenvironment inflammation (FIGS. 4 and 5). However, to address how Cd200 causes these effects requires direct genetic manipulation.

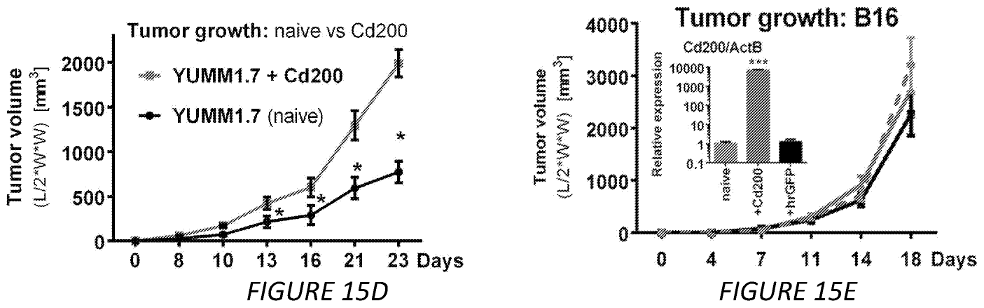

We used lentiviral delivery of mouse Cd200 cDNA (pLenti6.3) into YUMM1.7 cells (FIG. 15). To verify increased Cd200 expression, we used Western blot and ELISA of the culture supernatant (FIG. 15A/B). Helping to explain the observed bands is recent data in B-cell chronic lymphoblastic leukemia (B-CLL) suggesting that Cd200 also exists as a shed, shorter form protein [56]. Functionally, Cd200 over-expression did not alter YUMM1.7 (or B16) in vitro cell growth rates, nor did it significantly alter the inherent NAD+/NADH ratios (FIG. 15C) or Pd-L1/L2 expression. However, after implantation in congenic C57Bl/6 mice, Cd200 overexpression resulted in accelerated tumor growth (FIG. 15D). Because tumor growth of OVA-modified B16 cells has been proposed to blunted by Cd200 over-expression [57], we tested this model as well, but without the forced immunogenicity. In contrast, and in keeping with our hypothesis, over-expression of Cd200 in the poorly immunogenic B16 model did not affect tumor growth (FIG. 15E).

(2B-2) Measures of Immune Evasion and Cytolytic Activities—

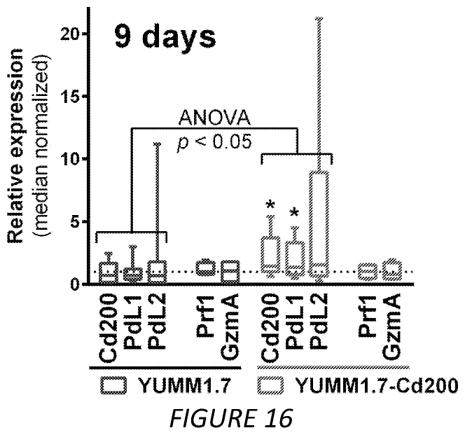

Throughout these experiments, at intermediate time points before tumor size differences are evident, we performed measurements of the associated cytolytic activity (using Prf1:GzmA), as well as presence of immunoediting, using PdL1:PdL2 (genes—Cd274:Pdcd1lg2) by qPCR (see FIG. 14B). Because Cd200 may recruit MDSCs, the expectation was to see changes in Pd-L1:L2 from increased immune evasion, but not in cytolytic genes, which is a measure of cell numbers (not activity) (FIG. 16).

(2C-1) Requirements for Cd200r1:

Because CD200 is thought to mediate its suppressive function through CD200R1 on receiving immune cells, we examine the effects of Cd200 on tumor growth in Cd200r1 nullizygous mice (on a C57/BL6 background) [58]. Specifically, modulation of Cd200 expressing using either over-expression, or shRNA mediated suppression, alters tumor growth, critically dependent on presence of Cd200R1 in the tumor bearing congenic host (FIG. 6).

### Example 3—the Role of CD200 in Response to Immune Checkpoint Therapies

The association between frequencies of predicted tumor neoantigens and efficacy of both anti-PD1 and anti-CTLA4 therapies suggests that the mutational makeup of cancers governs clinical responses [12, 13, 14].

Specifically, presence of neoantigens enables tumor immunoediting through an increased expression of molecules that can both immunize and tolerize against immune-associated cytolytic activity, including upregulation of PD-L1/L2 [59]. Hence, patients benefit from checkpoint therapies because of a “reset” balance between neoantigens and pro-/anti-immunogenic modifiers of T cells that surveil those. In this regard, CD200 promoting MDSCs of the innate immune system, may provide an important role in tumor escape (FIG. 2) and response to checkpoint therapy. Because inflamed immunoedited tumors are associated with better prognosis, we analyzed genomic data on response to anti-CTLA4 therapy in melanoma [26] (FIG. 5). We found a trend within the inflamed immunoedited tumors that low CD200 expression was associated with response to checkpoint therapy, yet statistical significance was not achieved due to the small cohort arm (n=6). Supported by this trend, and general outcome data (FIG. 4C), it is believed that CD200 affects anti-PD1 treatment and CD200 stratifies immune checkpoint therapy benefit.

### Example 4—CD200 Expression and Response to Immunotherapy

We established YUMM1.7-CD200 cells with accelerated tumor growth associated with an elevated frequency of MDSCs. Because MDSCs are able to suppress various T cell functions, it may be that tumors use CD200 enlist these cells to evade immune surveillance.

(4A-1) Altering the Response to Immunotherapy Through CD200 Modulation:

Parental or YUMM1 overexpressing Cd200 or shRNA (or Cas9/CRISPR) silencing nucleic acids or controls are subcutaneously inoculated on the flank areas of mice. At measurable tumor size (using caliper measurements), anti-PD1 (clone RMP1-14: BioXcell), anti-CTLA4, or mAb isotype control treatment is administrated (e.g., i.p. 25 mg twice weekly in PBS). Tumor measurements are performed twice weekly relative to mock, and until tumors reach >1.5 cm, when the experiment is terminated. Survival based on time to 1000 mm³ tumor.

(4A-2) Assessing Alterations and Function of Tumor-Infiltrating Immune Cells Following Treatment:

Because anti-PD-1 is expected to relieve tumor microenvironment-associated PD-L1 antagonism on PD1-bearing CD8 T cells, we measure the relative frequencies of CD45, CD4 (and subsets thereof: i.e. Tregs), CD8, NK, and MDSCs (FIGS. 14A-B) in the beginning as well as at the end of the longitudinal tumor treatment arms. In addition, using qPCR we measure the levels of cytolytic activity using GzmA/Prf1.

These experiments demonstrate whether CD200 expression reduces the efficacy of anti-PD1 or anti-CTLA4 checkpoint treatment due to an increased immune-suppressive microenvironment. Specifically, over-expression as well as genetic suppression of CD200 in tumor cells may affect efficacy measured as tumor size reduction [64] and innervation of cytolytic activity. We have chosen to take a genetic approach to these experiments because of the current uncertainty concerning CD200 biology and the potential roles played by the membrane bound vs soluble forms in modulating immune responses. Specifically, since we demonstrated an increase in soluble CD200 upon overexpression (FIG. 15A), neutralizing antibodies, although rapidly translatable to the clinic, may have lower than expected anti-tumor activity. The inducible expression/suppression of CD200 is designed as a means to prove the value of therapeutic targeting, either direct (using antibodies), or using metabolic or epigenetic targeted drugs.

### Example 5—Effect of Overexpression of CD200 on Tumor Response to Treatment with Checkpoint Inhibitors

YUMM1.7 mouse melanoma cells were modified with Cd200 over-expression or vector control and then subcutaneously implanted (about 200,000 cells/site) in C57Bl/6 mice (two sites per mice, n=7-9 across cohorts). When established tumors had reached an average size of 100 mm³ (D*D*L/2), once weekly treatment with anti-PD1 (RMP1-14; 250 μg) and anti-CTLA4 (9D9; 100 μg) started (versus IgG). Change in tumor size was monitored by caliper measurements twice weekly. Statistically significant differences assessed using multiple t-tests, with Holm-Sidak correction.

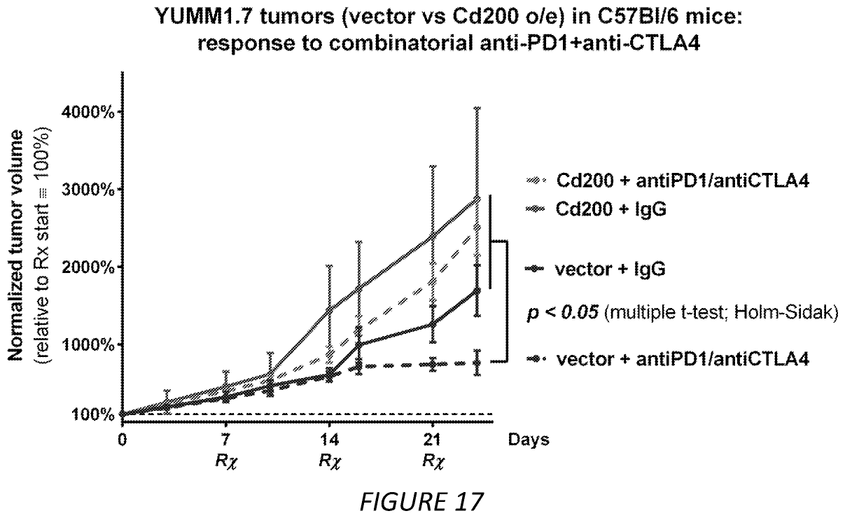

The results, shown in FIG. 17, show that increased levels of Cd200 endowed tumors with the ability to circumvent immune checkpoint inhibitor treatment.

### REFERENCES

1. Lito P, Rosen N, Solit D B. (2013) Tumor adaptation and resistance to RAF inhibitors. Nat Med. 19: 1401-1409.
2. Drake C G, Lipson E J, Brahmer J R. (2014) Breathing new life into immunotherapy: review of melanoma, lung and kidney cancer. Nat Rev Clin Oncol. 11:24-37.
3. Wolchok J D, Chan T A. (2014) Cancer: Antitumour immunity gets a boost. Nature 515: 496-498.
4. American Cancer Society (2016) Cancer Facts and Figures (cancer.org/research/cancerfactsstatistics/cancerfactsfigures2016/)
5. Hodi F S, O'Day S J, McDermott D F, Weber R W, Sosman J A, Haanen J B, Gonzalez R, Robert C, Schadendorf D, Hassel J C, Akerley W, van den Eertwegh A J, Lutzky J, Lorigan P, Vaubel J M, Linette G P, Hogg D, Ottensmeier C H, Lebbe C, Peschel C, Quirt I, Clark J I, Wolchok J D, Weber J S, Tian J, Yellin M J, Nichol G M, Hoos A, Urba W J. (2010) Improved survival with ipilimumab in patients with metastatic melanoma. N Engl J Med. 363: 711-723.
6. Schadendorf D, Hodi F S, Robert C, Weber J S, Margolin K, Hamid O, Patt D, Chen T T, Berman D M, Wolchok J D. (2015) Pooled Analysis of Long-Term Survival Data From Phase II and Phase III Trials of Ipilimumab in Unresectable or Metastatic Melanoma. J Clin Oncol. 33: 1889-1894.
7. Wolchok J D. (2015) PD-1 Blockers. Cell 162: 937.
8. Robert C, Schachter J, Long G V, Arance A, Grob J J, Mortier L, Daud A, Carlino M S, McNeil C, Lotem M, Larkin J, Lorigan P, Neyns B, Blank C U, Hamid O, Mateus C, Shapira-Frommer R, Kosh M, Zhou H, Ibrahim N, Ebbinghaus S, Ribas A; KEYNOTE-006 investigators. (2015) Pembrolizumab versus Ipilimumab in Advanced Melanoma. N Engl J Med. 372: 2521-2532.
9. Larkin J, Chiarion-Sileni V, Gonzalez R, Grob J J, Cowey C L, Lao C D, Schadendorf D, Dummer R, Smylie M, Rutkowski P, Ferrucci P F, Hill A, Wagstaff J, Carlino M S, Haanen J B, Maio M, Marquez-Rodas I, McArthur G A, Ascierto P A, Long G V, Callahan M K, Postow M A, Grossmann K, Sznol M, Dreno B, Bastholt L, Yang A, Rollin L M, Horak C, Hodi F S, Wolchok J D. (2015) Combined Nivolumab and Ipilimumab or Monotherapy in Untreated Melanoma. N Engl J Med. 373: 23-34.
10. Sharpe A H, Wherry E J, Ahmed R, Freeman G J. (2007) The function of programmed cell death 1 and its ligands in regulating autoimmunity and infection. Nat Immunol. 8: 239-245.
11. Cancer Genome Atlas Network. (2015) Genomic Classification of Cutaneous Melanoma. Cell 161: 1681-1696
12. Topalian S L, Hodi F S, Brahmer J R, Gettinger S N, Smith D C, McDermott D F, Powderly J D, Carvajal R D, Sosman J A, Atkins M B, Leming P D, Spigel D R, Antonia S J, Horn L, Drake C G, Pardoll D M, Chen L, Sharfman W H, Anders R A, Taube J M, McMiller T L, Xu H, Korman A J, Jure-Kunkel M, Agrawal S, McDonald D, Kollia G D, Gupta A, Wigginton J M, Sznol M. (2012) Safety, activity, and immune correlates of anti-PD-1 antibody in cancer. N Engl J Med. 366: 2443-2454.
13. Taube J M, Klein A P, Brahmer J R, Xu H, Pan X, Kim J H, Chen L, Pardoll D M, Topalian S L, Anders R A. (2014) Association of PD-1, PD-1 ligands, and other features of the tumor immune microenvironment with response to anti-PD-1 therapy. Clin Cancer Res. 20: 5064-5074.
14. Tumeh P C, Harview C L, Yearley J H, Shintaku I P, Taylor E J, Robert L, Chmielowski B, Spasic M, Henry G, Ciobanu V, West A N, Carmona M, Kivork C, Sej a E, Cherry G, Gutierrez A J, Grogan T R, Mateus C, Tomasic G, Glaspy J A, Emerson R O, Robins H, Pierce R H, Elashoff D A, Robert C, Ribas A. (2014) PD-1 blockade induces responses by inhibiting adaptive immune resistance. Nature 515: 568-571.
15. Hugo W, Zaretsky J M, Sun L, Song C, Moreno B H, Hu-Lieskovan S, Berent-Maoz B, Pang J, Chmielowski B, Cherry G, Sej a E, Lomeli S, Kong X, Kelley M C, Sosman J A, Johnson D B, Ribas A, Lo R S. (2016) Genomic and Transcriptomic Features of Response to Anti-PD-1 Therapy in Metastatic Melanoma. Cell 165: 35-44.
16. Gubin M M, Zhang X, Schuster H, Caron E, Ward J P, Noguchi T, Ivanova Y, Hundal J, Arthur C D, Krebber W J, Mulder G E, Toebes M, Vesely M D, Lam S S, Korman A J, Allison J P, Freeman G J, Sharpe A H, Pearce E L, Schumacher T N, Aebersold R, Rammensee H G, Melief C J, Mardis E R, Gillanders W E, Artyomov M N, Schreiber R D. (2014) Checkpoint blockade cancer immunotherapy targets tumour-specific mutant antigens. Nature 515: 577-581.
17. DuPage M, Mazumdar C, Schmidt L M, Cheung A F, Jacks T. (2012) Expression of tumour-specific antigens underlies cancer immunoediting. Nature 482: 405-409.
18. Tran E, Turcotte S, Gros A, Robbins P F, Lu Y C, Dudley M E, Wunderlich J R, Somerville R P, Hogan K, Hinrichs C S, Parkhurst M R, Yang J C, Rosenberg S A. (2014) Cancer immunotherapy based on mutation-specific CD4+ T cells in a patient with epithelial cancer. Science 344: 641-645.
19. Shankaran V, Ikeda H, Bruce A T, White J M, Swanson P E, Old L J, Schreiber R D. (2001) IFNgamma and lymphocytes prevent primary tumour development and shape tumour immunogenicity. Nature 410: 1107-1111.
20. Schreiber R D, Old L J, Smyth M J. (2011) Cancer immunoediting: integrating immunity's roles in cancer suppression and promotion. Science 331: 1565-1570.
21. Matsushita H, Vesely M D, Koboldt D C, Rickert C G, Uppaluri R, Magrini V J, Arthur C D, White J M, Chen Y S, Shea L K, Hundal J, Wendl M C, Demeter R, Wylie T, Allison J P, Smyth M J, Old L J, Mardis E R, Schreiber R D. (2012) Cancer exome analysis reveals a T-cell-dependent mechanism of cancer immunoediting. Nature 482: 400-404.
22. Rooney M S, Shukla S A, Wu C J, Getz G, Hacohen N. (2015) Molecular and genetic properties of tumors associated with local immune cytolytic activity. Cell 160: 48-61.
23. Rizvi N A, Hellmann M D, Snyder A, Kvistborg P, Makarov V, Havel J J, Lee W, Yuan J, Wong P, Ho T S, Miller M L, Rekhtman N, Moreira A L, Ibrahim F, Bruggeman C, Gasmi B, Zappasodi R, Maeda Y, Sander C, Garon E B, Merghoub T, Wolchok J D, Schumacher T N, Chan T A. (2015) Cancer immunology. Mutational landscape determines sensitivity to PD-1 blockade in non-small cell lung cancer. Science 348: 124-128.
24. van Rooij N, van Buuren M M, Philips D, Velds A, Toebes M, Heemskerk B, van Dijk L J, Behjati S, Hilkmann H, El Atmioui D, Nieuwland M, Stratton M R, Kerkhoven R M, Kesmir C, Haanen J B, Kvistborg P, Schumacher T N (2013). Tumor exome analysis reveals neoantigen-specific T-cell reactivity in an ipilimumab-responsive melanoma. J Clin Oncol. 31: e439-442.
25. Snyder A, Makarov V, Merghoub T, Yuan J, Zaretsky J M, Desrichard A, Walsh L A, Postow M A, Wong P, Ho T S, Hollmann T J, Bruggeman C, Kannan K, Li Y, Elipenahli C, Liu C, Harbison C T, Wang L, Ribas A, Wolchok J D, Chan T A (2014). Genetic basis for clinical response to CTLA-4 blockade in melanoma. N Engl J Med. 371: 2189-2199.
26. Van Allen E M, Miao D, Schilling B, Shukla S A, Blank C, Zimmer L, Sucker A, Hillen U, Geukes Foppen M H, Goldinger S M, Utikal J, Hassel J C, Weide B, Kaehler K C, Loquai C, Mohr P, Gutzmer R, Dummer R, Gabriel S, Wu C J, Schadendorf D, Garraway L A. (2015) Genomic correlates of response to CTLA4 blockade in metastatic melanoma. Science 350: 207-211.
27. Gorczynski R M. (2012) CD200:CD200R-Mediated Regulation of Immunity. ISRN Immunology. dx.doi.org/10.5402/2012/682168
28. Rygiel T P, Meyaard L. (2012) CD200R signaling in tumor tolerance and inflammation: A tricky balance. Curr Opin Immunol. 24: 233-238.
29. Webb M, Barclay A N. (1984) Localisation of the MRC OX-2 glycoprotein on the surfaces of neurones. J Neurochem. 43: 1061-1067.
30. Kretz-Rommel A, Qin F, Dakappagari N, Ravey E P, McWhirter J, Oltean D, Frederickson S, Maruyama T, Wild M A, Nolan M J, Wu D, Springhorn J, Bowdish K S. (2007) CD200 expression on tumor cells suppresses antitumor immunity: new approaches to cancer immunotherapy. J. Immunol. 178: 5595-5605.
31. Tonks A, Hills R, White P, Rosie B, Mills K I, Burnett A K, Darley R L. (2007) CD200 as a prognostic factor in acute myeloid leukaemia. Leukemia 21: 566-568.
32. Petermann K B, Rozenberg G I, Zedek D, Groben P, McKinnon K, Buehler C, Kim W Y, Shields J M, Penland S, Bear J E, Thomas N E, Serody J S, Sharpless N E. (2007) CD200 is induced by ERK and is a potential therapeutic target in melanoma. J Clin Invest. 117: 3922-3929.
33. Wong K K, Brenneman F, Chesney A, Spaner D E, Gorczynski R M. (2012) Soluble CD200 is critical to engraft chronic lymphocytic leukemia cells in immunocompromised mice. Cancer Res. 72: 4931-4943.
34. Barretina J, Caponigro G, Stransky N, Venkatesan K, Margolin A A, Kim S, Wilson C J, Lehar J, Kryukov G V, Sonkin D, Reddy A, Liu M, Murray L, Berger M F, Monahan J E, Morais P, Meltzer J, Korejwa A, Jane-Valbuena J, Mapa F A, Thibault J, Bric-Furlong E, Raman P, Shipway A, Engels I H, Cheng J, Yu G K, Yu J, Aspesi P Jr, de Silva M, Jagtap K, Jones M D, Wang L, Hatton C, Palescandolo E, Gupta S, Mahan S, Sougnez C, Onofrio R C, Liefeld T, MacConaill L, Winckler W, Reich M, Li N, Mesirov J P, Gabriel S B, Getz G, Ardlie K, Chan V, Myer V E, Weber B L, Porter J, Warmuth M, Finan P, Harris J L, Meyerson M, Golub T R, Morrissey M P, Sellers W R, Schlegel R, Garraway L A. (2012) The Cancer Cell Line Encyclopedia enables predictive modelling of anticancer drug sensitivity. Nature 483: 603-607.
35. Chen E Y, Tan C M, Kou Y, Duan Q, Wang Z, Meirelles G V, Clark N R, Ma′ayan A. (2013) Enrichr: interactive and collaborative HTML5 gene list enrichment analysis tool. BMC Bioinformatics 14: 128.
36. Jeffs A R, Glover A C, Slobbe L J, Wang L, He S, Hazlett J A, Awasthi A, Woolley A G, Marshall E S, Joseph W R, Print C G, Baguley B C, Eccles M R. (2009) A Gene Expression Signature of Invasive Potential in Metastatic Melanoma Cells. PLoS ONE 4: e8461.
37. Strub T, Giuliano S, Ye T, Bonet C, Keime C, Kobi D, Le Gras S, Cormont M, Ballotti R, Bertolotto C, Davidson I. (2011) Essential role of microphthalmia transcription factor for DNA replication, mitosis and genomic stability in melanoma. Oncogene 19: 2319-2332.
38. Garcia E, Marcos-Gutierrez C, del Mar Lorente M, Moreno J C, Vidal M. (1999) RYBP, a new repressor protein that interacts with components of the mammalian Polycomb complex, and with the transcription factor YY1. EMBO J. 18: 3404-3418.
39. Cunningham J T, Rodgers J T, Arlow D H, Vazquez F, Mootha V K, Puigserver P. (2007) mTOR controls mitochondrial oxidative function through a YY1-PGC-lalpha transcriptional complex. Nature 450: 736-740.
40. Blättler S M, Verdeguer F, Liesa M, Cunningham J T, Vogel R O, Chim H, Liu H, Romanino K, Shirihai O S, Vazquez F, Riiegg M A, Shi Y, Puigserver P. (2012) Defective mitochondrial morphology and bioenergetic function in mice lacking the transcription factor Yin Yang 1 in skeletal muscle. Mol Cell Biol. 32: 3333-3346.
41. Liu J, Cao L, Chen J, Song S, Lee I H, Quijano C, Liu H, Keyvanfar K, Chen H, Cao L Y, Ahn B H, Kumar N G, Rovira I I, Xu X L, van Lohuizen M, Motoyama N, Deng C X, Finkel T. (2009) Bmi 1 regulates mitochondrial function and the DNA damage response pathway. Nature 459: 387-392.
42. Chen Z, Marsden P A, Gorczynski R M. (2006) Cloning and characterization of the human CD200 promoter region. Mol Immunol. 43: 579-587.
43. Wegner M, Cao Z, Rosenfeld M G. (1992) Calcium-regulated phosphorylation within the leucine zipper of C/EBP beta. Science 256: 370-373.
44. Trautwein C, Caelles C, van der Geer P, Hunter T, Karin M, Chojkier M. (1993) Transactivation by NF-IL6/LAP is enhanced by phosphorylation of its activation domain. Nature 364: 544-547.
45. Yang B S, Hauser C A, Henkel G, Colman M S, Van Beveren C, Stacey K J, Hume D A, Maki R A, Ostrowski M C. (1996) Ras-mediated phosphorylation of a conserved threonine residue enhances the transactivation activities of c-Ets1 and c-Ets2. Mol Cell Biol. 16: 538-547.
46. Li J, Song J S, Bell R J, Tran T N, Haq R, Liu H, Love K T, Langer R, Anderson D G, Lame L, Fisher D E. (2012) YY1 regulates melanocyte development and function by cooperating with MITF. PLoS Genet. 8: e1002688.
47. Laurette P, Strub T, Koludrovic D, Keime C, Le Gras S, Seberg H, Van Otterloo E, Imrichova H, Siddaway R, Aerts S, Cornell R A, Mengus G, Davidson I. (2015) Transcription factor MITF and remodeller BRG1 define chromatin organisation at regulatory elements in melanoma cells. Elife 4.
48. Yasumoto K, Takeda K, Saito H, Watanabe K, Takahashi K, Shibahara S. (2002) Microphthalmia-associated transcription factor interacts with LEF-1, a mediator of Wnt signaling. EMBO J. 21: 2703-2714.
49. Goding C R (2000) Mitf from neural crest to melanoma: signal transduction and transcription in the melanocyte lineage. Genes Dev. 14: 1712-1728.
50. Liu R, Liu H, Chen X, Kirby M, Brown P O, Zhao K. (2001) Regulation of CSF1 promoter by the SWI/SNF-like BAF complex. Cell 106: 309-318.
51. Pratilas C A, Taylor B S, Ye Q, Viale A, Sander C, Solit D B, Rosen N. (2009) (V600E)BRAF is associated with disabled feedback inhibition of RAF-MEK signaling and elevated transcriptional output of the pathway. Proc Natl Acad Sci USA. 106: 4519-4524.
52. Cao X, Cai S F, Fehniger T A, Song J, Collins L I, Piwnica-Worms D R, Ley T J. (2007) Granzyme B and perforin are important for regulatory T cell-mediated suppression of tumor clearance. Immunity 27: 635-646.
53. Underwood J C. (1974) Lymphoreticular infiltration in human tumours: prognostic and biological implications: a review. Br J Cancer. 30: 538-548.
54. Hewitt H B, Blake E R, Walder A S. (1976) A critique of the evidence for active host defence against cancer, based on personal studies of 27 murine tumours of spontaneous origin. Br J Cancer 33: 241-259.
55. Ho P C, Meeth K M, Tsui Y C, Srivastava B, Bosenberg M W, Kaech S M. (2014) Immune-Based Antitumor Effects of BRAF Inhibitors Rely on Signaling by CD40L and IFNγ. Cancer Res. 74: 3205-3217.
56. Twito T, Chen Z, Khatri I, Wong K, Spaner D, Gorczynski R. (2013) Ectodomain shedding of CD200 from the B-CLL cell surface is regulated by ADAM28 expression. Leuk Res. 37: 816-821.
57. Talebian F, Liu J Q, Liu Z, Khattabi M, He Y, Ganju R, Bai X F. (2012) Melanoma cell expression of CD200 inhibits tumor formation and lung metastasis via inhibition of myeloid cell functions. PLoS One 7:e31442.
58. Boudakov I, Liu J, Fan N, Gulay P, Wong K, Gorczynski R M (2007). Mice lacking CD200R1 show absence of suppression of lipopolysaccharide-induced tumor necrosis factor-alpha and mixed leukocyte culture responses by CD200. Transplantation 84: 251-257.
59. Rooney M S, Shukla S A, Wu C J, Getz G, Hacohen N (2015). Molecular and genetic properties of tumors associated with local immune cytolytic activity. Cell 160: 48-61.
60. Vazquez F, Lim J H, Chim H, Bhalla K, Girnun G, Pierce K, Clish C B, Granter S R, Widlund H R, Spiegelman B M, Puigserver P. (2013) PGC1α expression defines a subset of human melanoma tumors with increased mitochondrial capacity and resistance to oxidative stress. Cancer Cell 23: 287-301.
61. Haq R, Shoag J, Andreu-Perez P, Yokoyama S, Edelman H, Rowe G C, Frederick D T, Hurley A D, Nellore A, Kung A L, Wargo J A, Song J S, Fisher D E, Arany Z, Widlund H R. (2013) Oncogenic BRAF regulates oxidative metabolism via PGC1α and MITF. Cancer Cell 23: 302-315.
62. Davies H, et al. (2002) Mutations of the BRAF gene in human cancer. Nature 417: 949-954.
63. Frederick D T, Piris A, Cogdill A P, Cooper Z A, Lezcano C, Ferrone C R, Mitra D, Boni A, Newton L P, Liu C, Peng W, Sullivan R J, Lawrence D P, Hodi F S, Overwijk W W, Lizee G, Murphy G F, Hwu P, Flaherty K T, Fisher D E, Wargo J A. (2013) BRAF inhibition is associated with enhanced melanoma antigen expression and a more favorable tumor microenvironment in patients with metastatic melanoma. Clin Cancer Res. 19: 1225-1231.
64. Spranger S, Bao R, Gajewski T F. (2015) Melanoma-intrinsic β-catenin signalling prevents anti-tumour immunity. Nature 523: 231-235.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method for determining whether a subject who has cancer is likely to benefit from treatment with a checkpoint inhibitor, the method comprising:
obtaining a sample comprising cancer cells from a subject;
evaluating the presence and/or level of CD200 in the sample;
comparing the presence and/or level of CD200 with a reference level, wherein a level of CD200 that is less than or equal to the reference level of CD200 indicates a high likelihood of response and a level of CD200 in a subject that is greater than the reference level of CD200 indicates a low likelihood of response, and
selecting and administering a treatment comprising a checkpoint inhibitor to a subject who has a level of CD200 that is less than or equal to the reference level of CD200.

2. The method of claim 1, wherein the sample comprising cancer cells is obtained by punch biopsy, needle biopsy, or tissue biopsy obtained during resection.

3. (canceled)

4. (canceled)

5. The method of claim 1, further comprising selecting a treatment comprising administration of a checkpoint inhibitor and one or both of a MEK inhibitor and/or a BRAF inhibitor to a subject who has a level of CD200 that is greater than the reference level of CD200.

6. The method of claim 3, further comprising administering the treatment comprising administration of a checkpoint inhibitor and one or both of a MEK inhibitor and/or a BRAF inhibitor to a subject who has a level of CD200 that is greater than the reference level of CD200.

7. The method of claim 1, wherein evaluating the presence and/or level of CD200 in the sample comprises determining a level of CD200 mRNA in the sample.

8. The method of claim 1, wherein the subject has melanoma, neuroblastoma, small cell lung carcinoma, mesothelioma, retinoblastoma, glioma, medulloblastoma, and ganglioneuroma.

9. The method of claim 8, wherein the subject has melanoma.

10. The method of claim 1, further comprising determining whether the cancer is characterized by a high degree of immune infiltration, by
(i) detecting the presence of NK, MDSC, CD4+, CD3+ and/or CD8+ immune cells;
(ii) detecting the presence of PDL1 (programmed cell death ligand 1: CD274), PDL2 (programmed cell death ligand 2: PDCD1LG2), granzyme A (GMZA) and/or perforin transcripts (PRF1); and/or
(iii) detecting the presence of PDL1, PDL2, granzyme A and/or perforin protein or activity, wherein the detection of the presence of NK, MDSC, CD4+, CD3+ and/or CD8+ immune cells above a threshold; and/or levels or presence of PDL1, PDL2, granzyme A and/or perforin mRNA or protein above a reference level indicates the presence of immune infiltration and a high likelihood of response to checkpoint inhibitors, and levels or presence of PDL1, PDL2, granzyme A and/or perforin mRNA or protein above a reference level indicates the absence of immune infiltration and a low likelihood of response to checkpoint inhibitors.

11. The method of claim 10, further comprising selecting a treatment comprising administration of a checkpoint inhibitor for a subject who has (i) NK, MDSC, CD4+CD3+ and/or CD8+ immune cells above a threshold; and/or levels or presence of PDL1, PDL2, granzyme A and/or perforin mRNA or protein above a reference level, and (ii) a level of CD200 that is less than or equal to the reference level of CD200.

12. The method of claim 11, further comprising administering the treatment comprising administration of a checkpoint inhibitor to a subject who has (i) NK, MDSC, CD4+, CD3+ and/or CD8+ immune cells above a threshold; and/or levels or presence of PDL1, PDL2, granzyme A and/or perforin mRNA or protein above a reference level, and (ii) a level of CD200 that is less than or equal to the reference level of CD200.

13. The method of claim 10, further comprising selecting a treatment comprising administration of a checkpoint inhibitor and a MEK inhibitor to a subject who has (i) NK, MDSC, CD4+, CD3+ and/or CD8+ immune cells above a threshold; and/or levels or presence of PDL1, PDL2, granzyme A and/or perforin mRNA or protein above a reference level, and (ii) a level of CD200 that is greater than the reference level of CD200.

14. The method of claim 13, further comprising administering the treatment comprising administration of a checkpoint inhibitor and a MEK inhibitor to a subject who has (i) NK, MDSC, CD4+, CD3+ and/or CD8+ immune cells above a threshold; and/or levels or presence of PDL1, PDL2, granzyme A and/or perforin mRNA or protein above a reference level, and (ii) a level of CD200 that is greater than the reference level of CD200.

15. The method of claim 13, further comprising selecting a treatment that does not comprise administration of a checkpoint inhibitor to a subject who has levels of NK, MDSC, CD4+, CD3+ and/or CD8+ immune cells below a threshold; and/or levels of PDL1, PDL2, granzyme A and/or perforin mRNA or protein below a reference level.

16. The method of claim 15, wherein the selected treatment comprises one or both of a MEK inhibitor and/or a BRAF inhibitor.

17. A method for treating a subject with cancer, the method comprising administering to the subject a therapeutically effective amount of a checkpoint inhibitor and a MEK inhibitor.

18. The method of claim 5, wherein the MEK inhibitor is selected from the group consisting of trametinib, cobimetanib, Binimetinib (MEK162), Selumetinib, PD-325901, CI-1040, PD035901, U0126-EtOH, PD184352 (CI-1040), TAK-733, PD98059, PD318088, BI-847325, GDC-0623, APS-2-79 HCl, Myricetin, Honokiol, SL-327, Refametinib (RDEA119, Bay 86-9766), BIX 02189, BIX 02188, AZD8330, TAK-733, and Pimasertib; and/or wherein the BRAF inhibitor is selected from the group consisting of BMS-908662, R05212054 (also known as RG7256 or PLX3603), GDC-0879, PLX-4720, GSK2118436, sorafenib tosylate, LGX818, vemurafenib, dabrafenib, encorafenib, or RAF265.

19. The method of claim 1, wherein the checkpoint inhibitor is an antibody, preferably selected from the group consisting of anti-CD137; anti-PD-1 (programmed cell death 1); anti-PDL1; anti-PDL2; and anti-CTLA-4.

20.-24. (canceled)

25. The method of claim 17, wherein the MEK inhibitor is selected from the group consisting of trametinib, cobimetanib, Binimetinib (MEK162), Selumetinib, PD-325901, CI-1040, PD035901, U0126-EtOH, PD184352 (CI-1040), TAK-733, PD98059, PD318088, BI-847325, GDC-0623, APS-2-79 HCl, Myricetin, Honokiol, SL-327, Refametinib (RDEA119, Bay 86-9766), BIX 02189, BIX 02188, AZD8330, TAK-733, and Pimasertib; and/or wherein the BRAF inhibitor is selected from the group consisting of BMS-908662, R05212054 (also known as RG7256 or PLX3603), GDC-0879, PLX-4720, GSK2118436, sorafenib tosylate, LGX818, vemurafenib, dabrafenib, encorafenib, or RAF265.

26. The method of claim 25, wherein the checkpoint inhibitor is an antibody, preferably selected from the group consisting of anti-CD137; anti-PD-1 (programmed cell death 1); anti-PDL1; anti-PDL2; and anti-CTLA-4.
